(19) United States
(12) Patent Application Publication
(10) Pub. No.: US 2025/0176637 A1
(43) Pub. Date: Jun. 5, 2025

(54) Aerosol Generation Device

(71) Applicant: JT International SA, Geneva (CH)

(72) Inventors: Olayiwola Olamiposi Popoola, Walton-on-Thames (GB); Kyle Adair, Lisburn (GB); Peter Loveday, Epsom (GB)

(73) Assignee: JT International SA, Geneva (CH)

(21) Appl. No.: 18/832,177

(22) PCT Filed: Jan. 24, 2023
(86) PCT No.: PCT/EP2023/051652

§ 371 Date: Jul. 23, 2024

(30) Foreign Application Priority Data
Jan. 27, 2022 (EP) 22153648.5

(51) Int. Cl. A24F 40/51; A24F 40/53; A24F 40/57; H05B 1/02

(52) U.S. Cl. CPC A24F 40/51; A24F 40/53; A24F 40/57; H05B 1/0252

(57) ABSTRACT

An aerosol generation device for generating an aerosol or vapor includes: an orientation sensor for sensing an orientation of the device, and a controller for: detecting a first set of one or more states of the device, the first set defining a sequence of states, and the first set including at least one state detected based at least in part on the sensed orientation; determining, based at least in part on the first set of states, a first operation of the aerosol generation device to be performed; controlling the aerosol generation device in accordance with the first operation; and causing a memory to store the first set of states and the first operation in association with each other, the association being for controlling a subsequent operation of the aerosol generation device when a subsequently detected second set of states of the aerosol generation device matches the first set of states.

### TECHNICAL FIELD

Example aspects herein relate to aerosol generation from a consumable, and in particular to an aerosol generation device, a controller for an aerosol generation device, a method for controlling an aerosol generation device, and computer program.

### BACKGROUND

There are known devices used to heat or warm aerosolisable substances, in order to generate an aerosol, which are portable and hand-held. For example, aerosol generation devices, with known types such as atomizers, vaporizers, electronic cigarettes, e-cigarettes, cigalikes, etc. are used to heat aerosolisable substances as a reduced-risk or modified-risk device from conventional tobacco products.

### SUMMARY OF THE DISCLOSURE

As aerosol generation devices are intended to be used by persons, their user friendliness is an important aspect of these devices. The present inventors have recognised that users are likely to have or form habits when using aerosol generation devices, but that conventional aerosol generation device may not be configurable to match the user's habits, or it may require the user to configure manually the device, which is cumbersome to the user.

At the same time, aerosol generation devices are significantly affected by the environment or the situation in which they operate, yet these devices have generally little to no means to reduce these effects, leading to an inconsistent user experience.

There is therefore a need to improve user friendliness of a device and in particular to facilitate how the aerosol generation device can operate in accordance with the user's preferences, in a broader range of situation.

The inventors have recognised the user's habits can be used to control how the device should operate in future uses, without requiring the user to indicate their preferences, thus improving the user friendliness of the aerosol generation device, and that the effects of environment can be countered by gaining a level of “situational awareness”, and using this awareness control the operation of the device.

It was appreciated that information relating to an observable or latent variable indicating a condition or a situation of the aerosol generation device can be obtained to adapt the operation of the aerosol generalisation device, and to recognise repeated patterns in the use of the aerosol generation device.

It was appreciated in particular that the orientation of the device is a factor which can be used to recognise repeated patterns in the use of the aerosol generation device whilst providing useful information about the situation in which the device is operating, as the orientation of the device may influence the generation of aerosol and/or other functionalities of the device. This provides for a more homogenous performance of the device.

According to a first example aspect disclosed herein, there is provided an aerosol generation device for generating an aerosol or vapor, the aerosol generation device comprising: an orientation sensor for sensing an orientation of the aerosol generation device, and a controller. The controller is for: detecting a first set of one or more states of the aerosol generation device, the first set preferably defining a sequence of states, and the first set comprising at least one state detected based at least in part on the sensed orientation; determining, based at least in part on the first set of states, a first operation of the aerosol generation device to be performed; controlling the aerosol generation device in accordance with the first operation; and causing a memory to store the first set of states and the first operation in association with each other, the association being for controlling a subsequent operation of the aerosol generation device when a subsequently detected second set of states of the aerosol generation device matches the first set of states.

According to another example aspect disclosed herein, there is provided a controller for an aerosol generation device, the aerosol generation device being for generating an aerosol or vapor and comprising an orientation sensor for sensing an orientation of the aerosol generation device. The controller is for: detecting a first set of one or more states of the aerosol generation device, the first set preferably defining a sequence of states, and the first set comprising at least one state detected based at least in part on the sensed orientation; determining, based at least in part on the first set of states, a first operation of the aerosol generation device to be performed; controlling the aerosol generation device in accordance with the first operation; and causing a memory to store the first set of states and the first operation in association with each other, the association being for controlling a subsequent operation of the aerosol generation device when a subsequently detected second set of states of the aerosol generation device matches the first set of states.

According to another example aspect disclosed herein, there is provided a method for controlling an aerosol generation device, the aerosol generation device being for generating an aerosol or vapor and comprising an orientation sensor for sensing an orientation of the aerosol generation device. The method comprises: detecting a first set of one or more states of the aerosol generation device, the first set preferably defining a sequence of states, and the first set comprising at least one state detected based at least in part on the sensed orientation; determining, based at least in part on the first set of states, a first operation of the aerosol generation device to be performed; controlling the aerosol generation device in accordance with the first operation; and causing a memory to store the first set of states and the first operation in association with each other, the association being for controlling a subsequent operation of the aerosol generation device when a subsequently detected second set of states of the aerosol generation device matches the first set of states.

According to another example aspect disclosed herein, there is provided a computer program comprising instructions which, when executed by one or more processor, cause the one or more processors to perform the method as summarized above.

According to another example aspect disclosed herein, there is provided an aerosol generation device for generating an aerosol or vapor. The aerosol generation device comprises: an orientation sensor for sensing an orientation of the aerosol generation device; a memory for storing a machine learning model having, as an input, a set of states of the aerosol generation device, and as an output, at least one operation of the aerosol generation device to be performed; and a controller. The controller is for: detecting a set of states of the aerosol generation device, the detected set of states preferably defining a sequence of states, and the detected set of states comprising at least one state detected based at least in part on the sensed orientation; determining, based at least in part on the detected set of states, an operation of the aerosol generation device to be performed; controlling an operation of the aerosol generation device in accordance with the determined operation; and causing the machine learning model to learn an association between the detected set of states and the determined operation.

Although the above summarised aspects relate in particular to sensing the orientation of the aerosol generation device, another observable variable, or another latent variable indicating a condition or a situation of the aerosol generation device can be obtained instead, or in addition thereto, to adapt the operation of the aerosol generalisation device.

Thus, according to another example aspect disclosed herein, there is provided an aerosol generation device for generating an aerosol or vapor, the aerosol generation device comprising: a sensor for sensing an observable related to the aerosol generation device, and a controller. The controller is for: detecting a first set of one or more states of the aerosol generation device, the first set preferably defining a sequence of states, and the first set comprising at least one state detected based at least in part on the sensed observable; determining, based at least in part on the first set of states, a first operation of the aerosol generation device to be performed; controlling the aerosol generation device in accordance with the first operation; and causing a memory to store the first set of states and the first operation in association with each other, the association being for controlling a subsequent operation of the aerosol generation device when a subsequently detected second set of states of the aerosol generation device matches the first set of states.

According to another example aspect disclosed herein, there is provided a controller for an aerosol generation device, the aerosol generation device being for generating an aerosol or vapor and comprising a sensor for sensing an observable related to the aerosol generation device. The controller is for: detecting a first set of one or more states of the aerosol generation device, the first set preferably defining a sequence of states, and the first set comprising at least one state detected based at least in part on the sensed observable; determining, based at least in part on the first set of states, a first operation of the aerosol generation device to be performed; controlling the aerosol generation device in accordance with the first operation; and causing a memory to store the first set of states and the first operation in association with each other, the association being for controlling a subsequent operation of the aerosol generation device when a subsequently detected second set of states of the aerosol generation device matches the first set of states.

According to another example aspect disclosed herein, there is provided a method for controlling an aerosol generation device, the aerosol generation device being for generating an aerosol or vapor and comprising a sensor for sensing an observable related to the aerosol generation device. The method comprises: detecting a first set of one or more states of the aerosol generation device, the first set preferably defining a sequence of states, and the first set comprising at least one state detected based at least in part on the sensed observable; determining, based at least in part on the first set of states, a first operation of the aerosol generation device to be performed; controlling the aerosol generation device in accordance with the first operation; and causing a memory to store the first set of states and the first operation in association with each other, the association being for controlling a subsequent operation of the aerosol generation device when a subsequently detected second set of states of the aerosol generation device matches the first set of states.

According to another example aspect disclosed herein, there is provided a computer program comprising instructions which, when executed by one or more processor, cause the one or more processors to perform the method as summarized above.

According to another example aspect disclosed herein, there is provided an aerosol generation device for generating an aerosol or vapor. The aerosol generation device comprises: a sensor for sensing an observable related to the aerosol generation device; a memory for storing a machine learning model having, as an input, a set of states of the aerosol generation device, and as an output, at least one operation of the aerosol generation device to be performed; and a controller. The controller is for: detecting a set of states of the aerosol generation device, the detected set of states preferably defining a sequence of states, and the detected set of states comprising at least one state detected based at least in part on the sensed observable; determining, based at least in part on the detected set of states, an operation of the aerosol generation device to be performed; controlling an operation of the aerosol generation device in accordance with the determined operation; and causing the machine learning model to learn an association between the detected set of states and the determined operation.

### BRIEF DESCRIPTION OF THE DRAWINGS

Embodiments of the present invention, which are presented for better understanding the inventive concepts, but which are not to be seen as limiting the invention, will now be described with reference to the figures in which:

FIG. 1 is a schematic diagram of electrical components of an aerosol generation device;

FIG. 2 is a schematic view of an aerosol generation device in a three dimensional space;

FIGS. 3A to 3C are a schematic view showing sets of states of an aerosol generation device;

FIG. 4 are schematic views showing sets of states of an aerosol generation device; and

FIG. 5 shows a method for controlling an aerosol generation device.

### DETAILED DESCRIPTION

Although example embodiments will be described below, it will be evident that various modifications may be made to these example embodiments without departing from the broader spirit and scope of the invention. Accordingly, the following description and the accompanying drawings are to be regarded as illustrative rather than restrictive.

In the following description and in the accompanying figures, numerous details are set forth in order to provide an understanding of various example embodiments. However, it will be evident to those skilled in the art that embodiments may be practiced without these details.

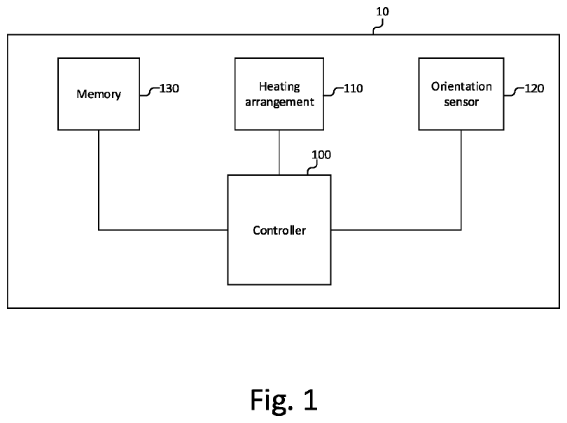

FIG. 1 is a schematic diagram of electrical components of an aerosol generation device 10 according to an example embodiment.

In the example shown on FIG. 1, the aerosol generation device 10 comprises a controller 100, an orientation sensor 110, a heating arrangement 120, and a memory 130.

As will be explained in more detail below, the controller 100 is arranged for controlling an operation of the aerosol generation device and for causing the memory 130 to store data for controlling subsequent operation(s) of the aerosol generation device. In some cases, the controller 100 may comprise one or more processor (e.g. a single/multiple core CPU, one or more microprocessors etc.), one or more working memories (e.g. random-access memory, RAM, flash memory etc.) and one or more non-volatile instructions stores (e.g. read-only memory (ROM), programmable ROM (PROM), erasable PROM (EPROM), electrically erasable PROM (EEPROM), flash memory, etc.) storing computer-readable instructions, whereby the processor(s) executing the computer-readable instructions in the instruction store(s) to control an operation of the aerosol generation device and cause the memory to store the date. In other examples, the controller 100 may be implemented as a hardware component including circuitry, such as integrated circuitry (IC), transmitting data to other elements of the aerosol generation device by way of communication channels (e.g. dedicated signal lines or bus), or by storing data in memory accessible to other elements.

The orientation sensor 110 is arranged for sensing an orientation of the aerosol generation device 10. The orientation sensor may be any sensor capable of obtaining an orientation of the aerosol generation device.

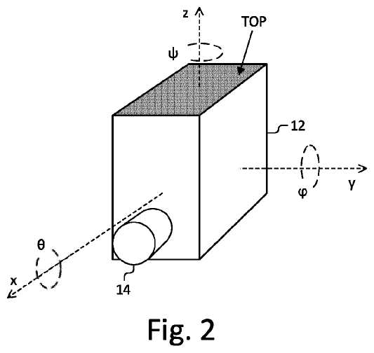

An explanation of the orientation of the aerosol generation device 10 will be provided with reference to FIG. 2, showing a schematic view of the aerosol generation device 10.

FIG. 2 shows an example of the aerosol generation device 10 oriented in a three dimensional space. It would be understood that the orientation of the aerosol generation device 10 can be defined in the three dimensional space as the rotation of the aerosol generation device from the frame of reference defined by the three orthogonal axes x, y and Z.

For sake of simplicity, in the example shown on FIG. 2, the aerosol generation device 10 is shaped such that a user is more inclined to hold the device in a particular way, with a specific side defined as the top, i.e. the side which is designed to be facing substantially up when the user uses the aerosol generation device to generate the aerosol while standing up (or sitting upright). In the example shown on FIG. 2, the aerosol generation device 10 comprises a body 12 and a mouthpiece 14, from which the user is to inhale the generated aerosol. Accordingly, the user would be inclined to hold the device when inhaling the aerosol generation device such that the side labelled ‘TOP’ is directed towards the top of the head of the user. However, it would be understood that this configuration is provided purely for simplicity, for the purpose of the description below, and the present invention is not limited to this configuration of the aerosol generation device 10.

Accordingly, the orientation sensor 110 is arranged for sensing the orientation of the aerosol generation device 10 and provide an indication of the sensed orientation to the controller 100. The orientation sensor 110 may be of any type of sensor capable of sensing an orientation of itself (and thus of the aerosol generation device 10, such as an accelerometer (e.g. MEMES accelerometer), a gyroscope (e.g. MEMS gyroscope), etc, and providing an indication of the sensed orientation to the controller 100, for example by outputting a signal.

Referring again to FIG. 1, the heating arrangement 120 is arranged for heating a substance, to generate an aerosol or vapor, using, for example, electrical power supplied from an electrical power source (not shown). The present invention is not limited to a particular type of substance, and any known type of substance suitable to generate an aerosol or vapor may be used, such as a fluid (liquid, gel), substrate (solid, paste type material in shredded, pelletized, powdered, granulated, strip or sheet form, or a combination thereof) etc. The substance may include tobacco, for example in dried or cured form, in some cases with additional ingredients for flavoring or producing a smoother or otherwise more pleasurable experience. In some cases, the heating arrangement 120 may comprises a heater for converting electrical power into thermal energy to heat the substance. It would be understood that the heater may be any type of heater suitable to heat the substance and generate the aerosol or vapor, such as conduction-based or convection-based heaters (e.g. a coil, a coil-and-wick combination), as the present invention is not limited in this aspect. The heating arrangement 120 may comprise additional elements, such as a temperature sensor, a power converter (e.g. a booster circuit) for converting the electrical power received from the electrical power source into an electrical power suitable to heat the substance.

The memory 130 is arranged for storing data for controlling operations of the aerosol generation device 10. The memory 130 may be any form of non-volatile instructions store (e.g. read-only memory (ROM), programmable ROM (PROM), erasable PROM (EPROM), electrically erasable PROM (EEPROM), flash memory, etc.). Although shown as a component separate from the controller 100, the controller 100 typically includes a memory storing computer-readable instructions, to be executed by a processor, and the memory of the controller may function as the memory 130.

The controller 100 is arranged for detecting states of the aerosol generation device 10. As used herein, a state of the aerosol generation device indicates a condition or a situation in which the aerosol generation device 10 is, and the state is defined by one or more parameters related to the aerosol generation device, such as its orientation, a temperature of a component of the aerosol generation device or the environment around the device, an amount of aerosolisable substance left, a state of charge of an electrical power source, etc. In other words, the controller 100 detects a state of the aerosol generation device by obtaining value(s) for each of the parameter(s) defining the state.

In some cases, a specific value of a parameter, or a specific range of values of a parameter may be defining the state. In these cases, the controller may detect the state if the obtained value corresponds to the specific value or falls within the specific range of values. The parameter(s) defining the state of the aerosol generation device 10 may be parameters that are controllable, by the controller 100 and/or by another controller in the aerosol generation device 10, or simply observable (e.g. by being sensed, or determined based on information gathered by the controller 100 or other component(s) of the aerosol generation device 10).

The controller 100 detects a set of one or more states of the aerosol generation device. The set of states includes a state which is defined based (at least in part) on the orientation of the aerosol generation device 10.

Preferably, the sets of states may define respective sequences of states (i.e. the controller 100 detects the set of states if the states are individually detected in a defined order).

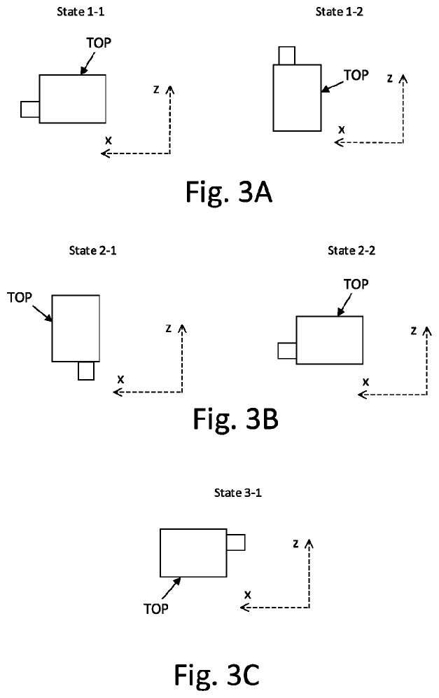

By way of non-limiting examples, the following Table 1 shows a number of sets of states of the aerosol generation device, which will be explained by reference to FIGS. 3A to 3D:

TABLE 1
Set of states

| Set | Definition |
|---|---|
| Set 1: Use while | State 1-1: orientation “TOP facing UP” + |
| standing up | aerosol generation start |
| | State 1-2: orientation “MOUTHPIECE facing |
| | UP, TOP facing SIDE” + aerosol |
| | generation end |
| Set 2: use while | State 2-1: orientation “MOUTHPIECE facing |
| laying down | DOWN, TOP facing SIDE” + aerosol |
| | generation start |
| | State 2-2: orientation “TOP facing UP” + |
| | aerosol generation end |
| Set 3: sleep mode 1 | State 3-1: orientation “TOP facing DOWN” |
| Set 4: sleep mode 2 | State 4-1: orientation “TOP facing DOWN” |
| | State 4-2: battery level low |
| | State 4-3: sleep timer expired |

The set 1 defines a sequence of the state 1-1 and state 1-2, which indicates a typical use of the aerosol generation device 10 while the user is standing up or sitting upright.

The controller detects state 1-1 as the top of the device is facing up (i.e. the top defines a substantially horizontal plane with the body 12 of the aerosol generation device 10 located substantially below the plane, as shown on the left side of FIG. 3A), and the generation of aerosol is started.

The controller 100 may detect the start of the aerosol generation based on an action of the user on the aerosol generation device. For example, the controller 100 may detect that the user has pressed a button or switch triggering the generation of aerosol, or the controller 100 may detect a pressure change in a heating chamber of the device indicating that the user has started inhaling from the mouthpiece. This may be determined based on a flow pressure measured in the aerosol generation device 10.

Then, the controller detects state 1-2 as the aerosol generation device 10 is substantially vertical, as shown on the right side of FIG. 3A, with the top of the device is facing towards the side (i.e. the top defines a substantially vertical plane) and the mouthpiece 14 is located above the body 12 of the aerosol generation device 10, and the generation of aerosol is ended.

The controller 100 may detect the end of the aerosol generation based on the user stopping the action on the aerosol generation device, for example, the controller 100 may detect that the user has stopped pressing the button or switch, or the controller 100 may detect a second pressure change in the heating chamber of the device indicating that the user has stopped inhaling from the mouthpiece.

In other words, as shown on FIG. 3A, the set of states 1 involves the start of the aerosol generation whilst the top of the aerosol generation device is facing up, and the controller detects a transition of the aerosol generation device 100 involving a rotation substantially in the ax-plane, together with the end of the aerosol generation.

The set 2 defines a sequence of the state 2-1 and state 2-2, which indicates a typical use of the aerosol generation device 10 while the user is laying down, or looking up.

As would be understood from the description of the set 1 above, the controller 100 may detect state 2-1 by detecting that the aerosol generation device 10 is substantially vertical, as shown on the left side of FIG. 3B, with the top of the device is facing towards the side (i.e. the top defines a substantially vertical plane) and the mouthpiece 14 is located below the body 12 of the aerosol generation device 10, and the generation of aerosol is started.

Then, the controller detects state 2-2 as the aerosol generation device 10 as the top of the device is facing up (i.e. the top defines a substantially horizontal plane with the body 12 of the aerosol generation device 10 located below the plane, as shown on the right side of FIG. 3B), and the generation of aerosol is ended.

It is appreciated that the performance of components of the aerosol generation device 10 (such as the heating arrangement, the battery etc.), or operations of the aerosol generation device (such as the heating of the aerosolisable substance, the vaporizing of particles, the supply of electric power to the heating arrangement or the supply of aerosol to mouthpiece) may depend on the orientation of the aerosol generation device 10. In particular, if components may move relative to one another, for example the aerosolisable substance moving relative the heating arrangement or the battery moves relative to the coupling to the electronic circuit of the aerosol generation device, variations in the performance of the aerosol generation device 10 may occur, leading to a reduced user experience. For example, in case of a device configured to generate an aerosol or vapor using a liquid in a container and a wick, the location of the liquid in the container, how it is provided to the wick, how the wick operates, etc. will depend greatly on the orientation of the device.

Therefore, by determining the orientation of the aerosol generation device 10, the controller 100 gains situational awareness of the aerosol generation device 10, which is used to improve the control of the aerosol generation device 10, as explained below.

In addition, releasable elements may have an increase of accidental release in certain orientations, which may lead to unexpected or undesirable operation. For example, a particular orientation may involve greater risks that a consumable including the aerosolisable substance gets uncoupled from a receptacle on the aerosol generation device, or that electrical coupling between the power source and the controller 100 may become unstable. Accordingly, having knowledge of the orientation of the aerosol generation device may improve the safe operation of the device.

Sets 3 and 4 will now be described, with reference to FIG. 3C.

Set 3 is a set comprising one state (state 3-1) and indicating that a use of the aerosol generation device 10 has stopped.

As shown on FIG. 3C, the controller 100 detects state 3-1 as the top of the device is facing down (i.e. the top defines a substantially horizontal plane with the body 12 of the aerosol generation device 10 located substantially above the plane).

The aerosol generation device 10 may be arranged to shift into an operation mode reducing the electrical power being consumed (e.g. a standby or sleep mode) when the user leaves the aerosol generation device with the top surface down. Accordingly, by detecting state 3-1, the controller 100 can determine that the aerosol generation device 10 may shift into the reduced power mode.

Set 4 is a set comprising three states, that do not define a sequence. In other words, the set 4 can be detected by detecting the three states in any given order.

State 4-1 corresponds to state 3-1 and its description will be omitted.

The controller 100 may detect state 4-2 if a state of charge of an electrical power source (e.g. a battery), whether internal to the aerosol generation device 10 or coupled thereto, is low. For example, the controller 100 may detect state 4-2 if the level of charge of a battery of the aerosol generation device 10 is below 20%.

The controller 100 may also be arranged to start a timer (e.g. a “sleep timer”) when the generation of the aerosol is stopped, and to detect state 4.3 if the generation of the aerosol is not re-started and the timer expires.

By detecting either of sets 3 or 4, the controller 100 can reduce the power consumption of the aerosol generation device 10, thus reducing the need of recharging the electrical power source, which in turn improves user experience.

It would be understood that using the alternative of set 3 would likely improve the reduction in power consumption, but may cause the user to experience delays each time the aerosol generation device 10 is picked-up again, as the aerosol generation device switches out of the reduced power consumption mode.

On the other hand, as set 4 is detected only when the battery level is low, and no aerosol has been generated for a given amount of time, the user would not experience delays if the aerosol generation is resumed before the timer expires or while the battery level is sufficiently high.

Referring back to FIG. 1, the controller 100 is arranged for determining, based at least in part on the detected set of states, an operation of the aerosol generation device 10 to be performed.

As used herein, the term operation indicates the control of a parameter of the aerosol generation device 10, which affect the generation of aerosol (e.g. initiating the supply of electrical power to heat the aerosolisable substance, initiating a timer to turn-off the supply of electrical power, interrupting the heating), setting a parameter or adjusting parameter(s) (e.g. a temperature depending on the electrical power being supplied, a voltage or a current of the electrical power provided to the heating arrangement 120), or another function of the aerosol generation device, such as providing information to the user via audio, visual and/or haptic feedback. Accordingly, the use of the term “operation” can be considered as “at least one” if multiple parameters are modified or a sequence of operations (e.g. turn off heater, then turn off display) are to be performed. The operation may be an operation desired by the user (e.g. generation of aerosol, change in rate of aerosol being generated, turning off the aerosol generation device), or it may be an operation automatically determined by the controller 100.

The controller 100 is arranged controlling the aerosol generation device 10 in accordance with the determined operation. The controller 100 may directly control of elements of the aerosol generation device 10, for example the state of switching element(s) allowing the supply of electrical power to the heating arrangement 120, or alternatively, the controller 100 may be communicatively coupled with another controller of the aerosol generation device 10 which in term controls the elements of the aerosol generation device. For example, a controller (e.g. a microcontroller, MCU) may be provided in the aerosol generation device to control the heating arrangement 120, which is separate from the controller 100. In some cases, the controller 100 may comprise a signal generator to generate one or more signals to control the elements of the aerosol generation device 10.

The controller 100 is arranged for causing the memory 130 to store a first detected set of states and the at least one operation determined based thereon in association with each other. The association allows the controller 100 to control a subsequent operation of the aerosol generation device 10 when it subsequently detects a subsequent set of states of the aerosol generation device which matches the first set of states.

Specifically, the controller 100 may either control the memory 130 to store data defining the detected set of states and data defining the determined operation in association with each other. Alternatively, the controller 100 may transmit the data defining the detected set of states and the determined operation to the memory with indication these should be stored in association with each other.

In some cases, the memory 130 may already be storing either the data defining the detected set of states or the data defining the determined operation, or both. In that case, the controller 100 may cause the memory 130 to store, the association between the detected set of states and the determined operation, as well as the data that is not yet stored in the memory, if any.

The memory 130 may store the data in a single table of a database or separate inter-connected tables. It would be clear to the skilled person that different ways of storing the data and the association can be implemented, as the present invention is not limited in this aspect. The memory 130 may store the data in any form allowing the controller 100 or any other entity reading the data to identify whether a subsequently detected set of state matches the set of state defined by the data, and determine the associated operation to be performed in case of a match.

The memory 130 is not limited to “one-to-one” associations between the set of states and the operations, may, in some case, store “one-to-many” associations where data defining a set of states is associated with data defining two or more operations, or the data defining an operation is associated with data defining two or more sets of states, which can be beneficial in terms of memory usage as a data defining an association is generally smaller than data defining a set of states or an operation.

Accordingly, the controller 100 may, when subsequently detecting a set of states, determine if the memory 130 stores data defining a matching set of states in association with data defining an operation. If so, the controller 100 may determine the associated operation as the operation to be performed, and control the operation of the aerosol generation device 10 accordingly.

The controller 100 may determine whether a detected set of states matches a stored set of states by comparing detected value(s) of each parameter(s) defining each detected state with corresponding value(s) of the parameter(s) defined by the data stored in the memory 130. In some cases, the controller 130 may determine a match only if all detected values of the parameters defining the state(s) correspond (i.e. an exact match). In other cases, variation(s) in some of the values of the parameters may be allowed, such as a variation in the orientation of the device (e.g. a difference of less than 10 degrees), the battery level, the amount of aerosolisable substance remaining, etc., with the allowable variation for each value stored in the memory 130 in association with the data defining the set of states. Alternatively, the controller 100 may be arranged to determine a stored set of state having the highest correspondence to the detected set of states (e.g. highest number of matching states) as the matching set of states (and accordingly control the aerosol generation device 10 in accordance with an operation stored with the matching set of states).

Preferably, the controller 100 may determine the operation of the aerosol generation device to be performed based on an association stored in the memory 130 between the detected set of states and the operation to be performed.

As explained above, the memory 130 may already be storing data defining a detected set of states in association with data defining an operation, either because the set of states was previously detected, or it is a predetermined set of states. Accordingly, the controller 100 may control the operation of the aerosol generation device 10 based on the data stored in the memory 130.

Preferably, the controller 100 may determine the operation of the aerosol generation device 10 to be performed based on a difference between a value of a parameter indicated by the detected set of states and a predetermined value of the parameter.

In some cases, the controller 100 may determine a value of a parameter of the aerosol generation device that is indicated by the detected set of states of the aerosol generation device 10 (e.g. by directly detecting the value or because the sequence of state indicate the value of the parameter). The controller 100 may determine that the detected value of the parameter differs from a predetermined value of the parameter. As a result, the controller 100 may determine an operation of the aerosol generation device to be performed based on the difference between the detected value and the predetermined value of the parameter. The operation to be performed may be used to reduce the determined difference, or to adapt an ongoing operation based on the difference.

For example, the aerosol generation device may comprise a thermal sensor for sensing a thermal characteristic of the aerosol generation device. The controller 100 may detect, from the sensed thermal characteristic, that a temperature in an aerosol chamber of the aerosol generation device is higher than a predetermined threshold and that an amount of aerosol in the chamber is lower than a predetermined threshold, indicating that the aerosolisable substance is not sufficiently in contact with the heating arrangement 120. Accordingly, the controller 100 may reduce the supply of electrical power to the heating arrangement, to avoid an increase in the temperature of the aerosol chamber.

As another example, the heating arrangement 120 may comprise two or more heaters located at a distance from each other, each to be in contact with and heat the aerosolisable substance. In use, the controller 100 may determine that the temperature of a first one of the heaters, (or the electrical power consumed by a first one of the heaters) is higher than another heater. The controller 100 may determine that the first heater is either defective or is no longer or not sufficiently in contact with the aerosolisable substance. As a result, the controller 100 may determine, as the operation to be performed, that the electric power supplied to the first heater is to be reduced or stopped.

In either of these examples, the controller 100 may, based on an orientation of the aerosol generation device 10 detected by the orientation sensor 110, determine that the detected orientation risk causing a malfunction (e.g. if the aerosolisable substance is not sufficiently in contact with the heating arrangement 120), interrupt the supply of electrical power to the heating arrangement, and notify the user that the aerosolisable substance should be re-filled, or a consumable or container including the aerosolisable substance should be replaced.

In some cases, the controller 100 may, in some cases, be arranged for interrupting the operation of the aerosol generation device 10 when the sensed orientation is substantially along a predetermined orientation.

Accordingly, the controller 100 may ensure that the aerosol generation device 10 operates without deviating from a predetermined value or range of values of certain parameters, improving the consistency in the operation of the aerosol generation device and as a result providing a more consistent user experience.

In some cases, the controller 100 may detect a state of the set of states based at least in part on a user action (e.g. a manipulation of the device or an input provided to the device) indicating the first operation. For example, the user pressing a button or switch as explained in connection with state 1-1 above indicates the operation of generating the aerosol.

Accordingly, storing the detected set of states in association with the indicated operation would allow the controller 100 to predict, when detecting the set of states again, what operation will likely be indicated by the user, allowing the controller 100 to adapt the operation accordingly, before the user indicates the operation, therefore improving the responsiveness of the device.

Preferably, the memory is arranged for storing at least one predetermined set of states of the aerosol generation device. Each predetermined set comprises one or more respective states of the aerosol generation device. Preferably, each predetermined set also define a respective sequence of states. As used herein, the predetermined set(s) of states are sets that are stored in the memory before being detected by the controller 100 (e.g. before use of the device). The predetermined set(s) may correspond to patterns that are likely to be detected during the use of the device, such as those shown on Table 1 above.

Preferably, the memory is arranged for storing at least one predetermined operation of the aerosol generation device to be performed. Each predetermined operation is to be associated with a set of states stored in memory, which is either a set of states detected by the controller 100 during use of the aerosol generation device 10, or a predetermined set of states. The predetermined operation(s) may also be associated with more than one set of states, as explained above. Accordingly, during use of the aerosol generation device 10, as the memory 130 need only store associations between the predetermined sets of states and the predetermined operations, the operation of the aerosol generation device 10 can be adapted without causing a large increase in amount of stored data in memory 130, which is particularly useful for devices having limited memory space. However, the predetermined sets of states are not limited to being associable with predetermined operations, and may be associated with operations that are determined during use of the aerosol generation device 10. Similarly, predetermined operations may also be associated with sets of states determined during use of the aerosol generation device 10.

Preferably, the memory 130 is arranged to store at least one association between a respective predetermined set of states and a respective predetermined operation. The controller 100 is arranged for, when determining an operation of the aerosol generation device 10 to be performed, determine a predetermined set of states matching the detected set of states, and controlling the aerosol generation device to operate in accordance with the predetermined operation associated with the predetermined set of states. Alternatively, the predetermined set(s) of states may be associated during use of the aerosol generation device 10 with operation(s) indicated by the user. This facilitate the detection of the set of states, as the controller 100 already knows the sets to be detected. Similarly, the predetermined operation(s) may be associated during use of the aerosol generation device with detected sets of states.

Accordingly, the controller 100 can automatically determine how to control the aerosol generation device 10 when the user starts using the aerosol generation device 10.

In the operation of the aerosol generation device 10, some parameters that can be controlled by the controller 100 have causal relationships with parameters that define detected states of the aerosol generation device 10. The controller 100 may hold, either in the memory 130 or elsewhere, information indicating causal relationships between a parameter that is controlled by the parameter and a parameter that is detected by the controller 100. Preferably, the controller 100 is arranged for determining a set of states stored in the memory 130 (a previously detected and stored set, or a predetermined set) that matches the detected set of states, and determining a difference between a detected state and a corresponding stored state. As explained above, the controller may consider the stored set of states which has the highest correspondence to the detected set of states is the matching set of states. When determining the match between the stored set of states and the detected set of states, the controller may determine a difference between a detected state and a stored state. Specifically, the controller 100 may determine a difference between a value of a parameter defining the state that is stored in memory, and the detected value of the parameter.

The controller 100 may determining a change in a controlled parameter of the aerosol generation device 10 that would cause the difference to decrease in a subsequent operation of the aerosol generation device 10. The controller 100 may use, for example, the information on causal relationships between controlled parameters and detected parameters to determine this change.

The controller 100 may then cause the memory to store data defining the change in the controlled parameter in association with a subset of the stored set of states. The stored subset includes states detected before the state in which the difference was determined.

Accordingly, the controller 100 may reduce differences in values of parameters, in subsequent operations of the aerosol generation device 10, and therefore improve consistency in operation of the aerosol generation device 10.

For example, assuming the memory 130 stores data defining the following sequence of states A:
State A-1: orientation “TOP facing UP”
State A-2: orientation “MOUTHPIECE facing DOWN/TOP facing SIDE”+aerosol generation start
State A-3: temperature of aerosol substance between 210° C. and 220° C.

The memory 130 stores, in association with the sequence of states A, data defining the following operation to heat the aerosol substance to a temperature of 230° C. to generate the aerosol:
upon detection of state A-3, increment the supply of electrical power by X mW for 5 seconds upon detection (X being any number)

In this example, the controller 100 detects states A-1 and A-2. However, the controller 100 detects the temperature of the aerosol substance to be 215° C. Accordingly, the controller 100 determines a difference of 5° C. between the stored values and the detected value. As a result, the increment of X mW for 5 seconds is likely to lead to a temperature of the aerosol substance notably higher than 230° C. which may deteriorate the aerosol substance, or the quality of the aerosol that is generated.

The controller 100 then determines, for example based on the stored information on causal relationship, that the supply of electrical power has a positive causal relationship with the temperature of the aerosol substance. As a result, the controller 100 determines that reducing the supply of electrical power (a change in operation) before detecting state A-3 should reduce the difference between the detected temperature and the stored temperature of the aerosol substance.

The controller 100 therefore cause the memory 130 to store data defining the change of operation (reduce the supply of electric power by Y mW) in association with data defining a subset of the sequence A, namely states A-1 and A-2.

Accordingly, in a subsequent operation of the aerosol generation device 10, the supply of electric power would be reduced when states A-1 and A-2 are detected, and the temperature of the aerosol substance is more likely to be near or closer to the stored range of values, therefore the aerosol generation device 10 would operate closer to the predetermined conditions.

Preferably, the controller 100 is arranged for detecting a movement of the aerosol generation device based on the sensed orientation. A movement of the aerosol generation device 10 may be a change or orientation (e.g. a rotation of the device about one or more axes), a translation of the aerosol generation device from one position to another (e.g. a displacement), or both. In these cases, the set of states comprises at least one state based on the detected movement.

Taking the exemplary set 1 shown on Table 1 above, state 1-2 may define a position of the aerosol generation device relative to the position of the aerosol generation device in state 1-1 (e.g. an translation in the upward direction indicating the user brings the aerosol generation device closer to his face). Accordingly, the controller 100 may detect a transition from state 1-1 to state 1-2 if the change in position (which may be detected based on an acceleration of the device sensed by the orientation sensor) is detected.

Preferably, the controller 100 is arranged for detecting an action of a user on the aerosol generation device. As used herein, an action of the user can be the user orienting the device in a specific way (TOP facing up, down, vertically, etc.), the user operating a button or switch on the device, the user inhaling the aerosol, the user removing or replacing an element of the aerosol generation device (e.g. the battery, a consumable or container comprising the aerosolisable substance), etc. The action is not necessarily based on the orientation (or change of orientation) of the device. In these cases, the controller 100 is arranged for detecting at least one state based on the detected action. Optionally, the action of the use may indicate a manipulation of the aerosol generation device, i.e. any movement or handling of the device that may be the result of a gesture of the user holding the device, and which can be determined based on the orientation of the device.

Preferably, the aerosol generation device 10 comprises the electrical power supply, and the controller 100 is arranged for controlling a supply of electrical power from the electrical power supply to the heating arrangement 120.

Preferably, the aerosol generation device 10 comprises a thermal sensor for sensing a thermal characteristic of the aerosol generation device and/or the aerosolisable substance. In these cases, the set of states detected by the controller 100 comprises at least one state based on a thermal value output from the thermal sensor.

Preferably, the controller 100 is arranged for determining an allowable range for the thermal value based on the sensed orientation. In these cases, the controller 100 may be arranged for interrupting the heating of the aerosolisable substance and/or the generation of aerosol, if the thermal value is determined not to fall within the allowable range. Alternatively, the controller 100 may be arranged to reduce the supply of electric power to the heating arrangement 120.

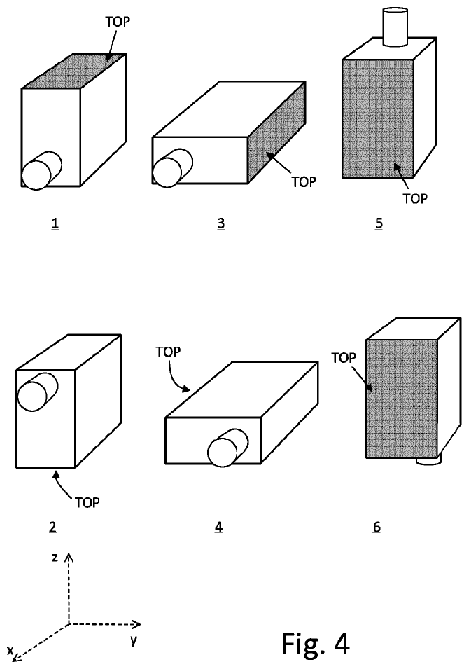

For example, the thermal value may be indicating a temperature of the aerosolisable substance. A first allowable range of the thermal value may be defined for when the aerosol generation device is oriented such that a mouthpiece of the aerosol generation extends horizontally or upward, and a second, different allowable range of the thermal value may be defined for when the mouthpiece extends downwards (see, for example FIG. 4, orientation 6). In the second case, the thermal value for the second allowable range may be set lower due to, for example, an increased safety of the user is provided who is located under the device, or because the heating arrangement 120 is in contact with more aerosolisable substance, etc.

Preferably, the aerosol generation device comprises a sensor for sensing at least one of an atmospheric pressure and an altitude of the aerosol generation device. In those cases, the controller 100 may be arranged to detect at least one state that is based (in part) on a sensed atmospheric pressure or altitude.

Accordingly, as the temperature at which a substance vaporises or sublimates depends on the pressure in the environment of the substance, the controller 100 can adapt the heating of the aerosolisable substance to reach the right temperature.

Preferably, the aerosol generation device comprises a power source (e.g. replaceable and/or rechargeable battery). In these cases, the controller 100 is arranged for detecting a state of the power source, and at least one state of the aerosol generation device is detected based on the state of the power source.

For example, the state of the power source may be the state of charge or level, previously described, or a stage in the life cycle of the power source, a characteristic of the power (e.g. voltage and/or current) output by the power source, etc. Accordingly, the controller 100 can better estimate how the power supplied by the power source will heat the aerosolisable substance.

Preferably, the controller 100 may be arranged to control the aerosol generation device in accordance with a determined operation by interrupting the operation of the aerosol generation device 10. Accordingly, if the detected set of state (in particular the orientation) indicate that the aerosol generation device or one of its components is at risk of malfunction, the use of the device or state of the aerosolisable substance cause safety risks, the operation may be interrupted to reduce, minimise or avoid the risks. In these cases, the controller 100 may notify the user, e.g. via visual, audio and/or haptic feedback that the operation is interrupted.

Preferably, the orientation sensor 110 comprise at least one of an accelerometer and/or a gyroscope, and sense the orientation based on data obtained from one or both of the accelerometer and gyroscope. However, it would be understood that the present invention is not limited in that aspect, and that any other sensor indicating an orientation of the aerosol generation device may be used instead of, or in addition to, the accelerometer and/or the gyroscope.

Preferably, the orientation sensor may be arranged to indicate one of six orientations of the aerosol generation device 10. As shown on FIG. 4, these orientations may be defined as: 1) the top of the device is facing up (the top defines a substantially horizontal plane with the device being substantially below the plane defined by the top); 2) the top of the device is facing down (the top defines a substantially horizontal plane with the device being substantially above the plane defined by the top); 3) the top of the device is facing to the side (the top defines a substantially vertical plane), and the mouthpiece is facing another side (the mouthpiece is along a substantially horizontal axis); 4) the top of the device is facing to the opposite side from orientation 3 (the top defines a substantially vertical plane), and the mouthpiece is facing another side (the mouthpiece is along a substantially horizontal axis); 5) the top is facing the side, with the mouthpiece facing up; and, 6) the top is facing the side, with the mouthpiece facing down. In these cases, the controller 100 may obtain from the orientation sensor an indication of which of the six orientation corresponds to the sensed orientation.

In some cases, the controller 100 can detect transitions from one of the orientation into the other based on a rotation of about 90 or about 180 degrees. For example, detecting a clockwise rotation in the yz-plane of about 90 degrees indicates a transition from orientation 1 to orientation 3 whereas a corresponding counter-clockwise rotation indicates a transition from orientation 1 to orientation 4.

In the examples described above, the controller 100 causes the memory to store sets of states and operations to be performed in association with each other. Alternatively, the memory 130 may store a machine learning model having, as an input, a set of states of the aerosol generation device, and as an output, at least one operation of the aerosol generation device to be performed.

For example, the controller 100 may detect the following sequence of states B of the aerosol generation device:
State B-1: orientation “TOP facing UP” (e.g. orientation 1 shown on FIG. 4)
State B-2: aerosol generation
State B-3 orientation “TOP facing UP”
State B-4: aerosol generation
State B-5: orientation “TOP facing UP”
State B-6: orientation “MOUTHPIECE facing DOWN, TOP facing SIDE” (e.g. orientation 6 shown on FIG. 4)
State B-7: aerosol generation
State B-8: orientation “MOUTHPIECE facing DOWN, TOP facing SIDE” (e.g. orientation 6 shown on FIG. 4)

This sequence of states indicates that the user is using the device while laying down (or looking up). Accordingly, if after state B-8, the controller 100 determines that the user is no longer using the device and the device transition into sleep mode, the sequence of states B can be used as indicative of a use session of the device, and that the device can transition into a reduced-power consumption mode once state B-8 is detected. As a result, the controller associates the sequence of state B with an operation to switch the aerosol generation device 10 into a reduced-power consumption mode, and cause the machine learning model in the memory 130 to learn this association.

The machine learning model may comprise any known type of machine learning model, including neural networks, support vector machines, linear regression, etc. as the present invention is not limited to a specific type of machine learning model.

The controller 100 may detect a set of states and determine an associated operation, independently from the machine learning model. For example, this may be determined based on the user's action or input provided to the device, based on a difference between a value of a parameter indicated by the detected set of states and a predetermined value of the parameter. In these cases, the controller 100 may cause the machine learning model in the memory 130 to learn (or reinforce) the association between the detected set of states and the determined operation.

By way of a non-limiting example, in an implementation, the controller 100 may have a system clock of 64 MHz or above, a volatile memory (e.g. RAM) of at least 96 kB, a non-volatile memory (e.g. ROM, EPROM, EEPROM, flash memory) of at least 512 kB to store programs to be executed, and a data bus having a speed of at least 400 kHz. In an embodiment, at least 10 kB of the non-volatile memory and 10 kB of the volatile memory may be allocated for the management of the feature extraction and features libraries for the machine learning model.

In addition, the controller 100 may be provided, before use, with a training dataset including pairs of sets of states and an associated operation, and cause the machine learning model to train (e.g. using a supervised training) using the training dataset.

The training dataset may be collected, for example, by collecting information on the state of the aerosol generation devices used by a number of users over time. After the data collection, sets of states may be obtained from the collected data, and each set of states may be associated with an operation, thus creating a training pair. Details of the generation of the training dataset will be omitted as they would become apparent to the skilled person from the above.

Preferably, the machine learning model trained with the training dataset may continue to learn associations between sets of states and operations that are determined by the controller 100. As each user is likely to form their own habit, this would allow the machine learning model to adapt to the habits of the user.

The machine learning model may be trained (with the training dataset and/or with sets of states and associated operations determined by the controller 100 during use) using any known training process, as the present invention is not limited in this regard.

It will be appreciated from the description above that certain example embodiments perform a method for controlling an aerosol generation device, the aerosol generation device being for generating an aerosol or vapor and comprising an orientation sensor for sensing an orientation of the aerosol generation device.

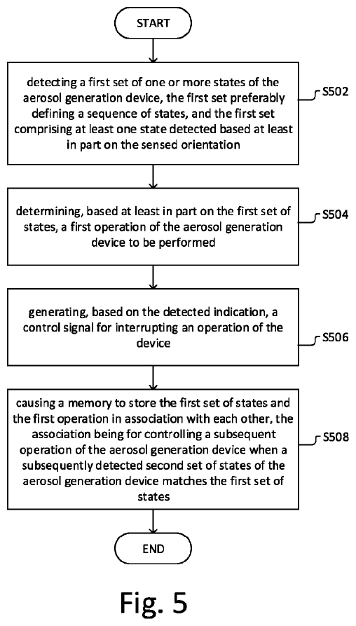

Referring to FIG. 5, at step 502, the aerosol generation device detects a first set of one or more states of the aerosol generation device, the first set preferably defining a sequence of states, and the first set comprising at least one state detected based at least in part on the sensed orientation.

At step 804, the aerosol generation device determines, based at least in part on the first set of states, a first operation of the aerosol generation device to be performed.

At step 806, the aerosol generation device operates in accordance with the first operation.

At step 808, the aerosol generation device causes a memory to store the first set of states and the first operation in association with each other, the association being for controlling a subsequent operation of the aerosol generation device when a subsequently detected second set of states of the aerosol generation device matches the first set of states.

### Modifications and Variations

Many modifications and variations can be made to the example embodiments described above.

In examples described above, the aerosol generation device comprises the electrical power supply (e.g. battery). However, this is not limiting as the electrical power supply may be external to the aerosol generation device instead.

In examples described above, the aerosol generation device comprises a temperature or thermal sensor, from which the controller 100 obtains a temperature of a component of the aerosol generation device 10 or the aerosolisable substance. Alternatively, the aerosol generation device may have a sensor measuring a current and/or voltage provided to the heating arrangement 120 or output from the electrical power source. In yet other cases, the controller 100 may be arranged to only obtain data from the orientation sensor 110 and detect the state of the device based on the sensed orientation.

In examples described above, the aerosol generation device 10 comprises an orientation sensor 110, from which the controller 100 obtains a sensed orientation of the aerosol generation device. Instead, the aerosol generation device 10 may comprise a sensor arranged for sensing another observable related to the aerosol generation device 10, the set of states detected by the controller 100 comprising at least one state detected based at least in part on the sensed observable.

By way of non-limiting examples, the observable may be indicating a movement of the aerosol generation device 10 from one position to the other (also obtainable by a sensor comprising an accelerometer and/or a gyroscope). For example, the controller 100 may detect that the aerosol generation device 10 moves upward after a period of motionlessness indicating the aerosol generation device 10 is picked up by the user, and the controller 100 may determine that the aerosol generation device 10 should be shifted out of the reduced power mode. Similarly, the controller 100 may detect a series of reciprocating upward and downward movements of the aerosol generation device 10 as indicating a use session of the aerosol generation device 10, and may determine the end of a typical use based on previously detected sequences of movements.

In other examples, the observable may be indicating an atmospheric pressure around the aerosol generation device 10, a state of the power source, a flow pressure, or any other observable described above, etc.

In addition, the controller 100 is not limited to controlling the aerosol generation device 10 based on the observable directly, and may instead determine a value of a latent variable from the sensed observable to control the aerosol generation device 10.

A person skilled in the art will, of course, recognize that modifications other than those described above can be made.

In particular, it would be understood that example embodiments described above may be combined.

In the foregoing description, example aspects are described with reference to several example embodiments. Accordingly, the specification should be regarded as illustrative, rather than restrictive. Similarly, the figures illustrated in the drawings, which highlight the functionality and advantages of the example embodiments, are presented for example purposes only. The architecture of the example embodiments is sufficiently flexible and configurable, such that it may be utilized in ways other than those shown in the accompanying figures.

Software embodiments of the examples presented herein may be provided as, a computer program, or software, such as one or more programs having instructions or sequences of instructions, included or stored in an article of manufacture such as a machine-accessible or machine-readable medium, an instruction store, or computer-readable storage device, each of which can be non-transitory, in one example embodiment. The program or instructions on the non-transitory machine-accessible medium, machine-readable medium, instruction store, or computer-readable storage device, may be used to program a computer system or other electronic device. The techniques described herein are not limited to any software configuration. They may find applicability in any computing or processing environment. The terms “computer-readable”, “machine-accessible medium”, “machine-readable medium”, “instruction store”, and “computer-readable storage device” used herein shall include any medium that is capable of storing, encoding, or transmitting instructions or a sequence of instructions for execution by the machine, computer, or computer processor and that causes the machine/computer/computer processor to perform any one of the methods described herein. Furthermore, it is common in the art to speak of software, in one form or another (e.g., program, procedure, process, application, module, unit, logic, and so on), as taking an action or causing a result. Such expressions are merely a shorthand way of stating that the execution of the software by a processing system causes the processor to perform an action to produce a result.

Some embodiments may also be implemented by the preparation of application-specific integrated circuits, field-programmable gate arrays, or by interconnecting an appropriate network of conventional component circuits.

Some embodiments include a computer program product. The computer program product may be a storage medium or media, instruction store(s), or storage device(s), having instructions stored thereon or therein which can be used to control, or cause, a computer or computer processor to perform any of the procedures of the example embodiments described herein. The storage medium/instruction store/storage device may include, by example and without limitation, an optical disc, a ROM, a RAM, an EPROM, an EEPROM, a DRAM, a VRAM, a flash memory, a flash card, a magnetic card, an optical card, nanosystems, a molecular memory integrated circuit, a RAID, remote data storage/archive/warehousing, and/or any other type of device suitable for storing instructions and/or data.

Stored on any one of the computer-readable medium or media, instruction store(s), or storage device(s), some implementations include software for controlling both the hardware of the aerosol generation device and for enabling the aerosol generation device or microprocessor to operate in accordance with the example embodiments described herein. Such software may include without limitation device drivers, operating systems, and user applications. Ultimately, such computer-readable media or storage device(s) further include software for performing example aspects of the invention, as described above.

Included in the programming and/or software of the aerosol generation device are software modules for implementing the procedures described herein. In some example embodiments herein, a module includes software, although in other example embodiments herein, a module includes hardware, or a combination of hardware and software.

While various example embodiments of the present invention have been described above, it should be understood that they have been presented by way of example, and not limitation. It will be apparent to persons skilled in the relevant art(s) that various changes in form and detail can be made therein. Thus, the present invention should not be limited by any of the above described example embodiments, but should be defined only in accordance with the following claims and their equivalents.

Further, the purpose of the Abstract is to enable the Patent Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the example embodiments presented herein in any way. It is also to be understood that any procedures recited in the claims need not be performed in the order presented.

While this specification contains many specific embodiment details, these should not be construed as limitations on the scope of any inventions or of what may be claimed, but rather as descriptions of features specific to particular embodiments described herein. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

In certain circumstances, multitasking and parallel processing may be advantageous. Moreover, the separation of various components in the embodiments described above should not be understood as requiring such separation in all embodiments.

Having now described some illustrative embodiments and embodiments, it is apparent that the foregoing is illustrative and not limiting, having been presented by way of example. In particular, although many of the examples presented herein involve specific combinations of apparatus or software elements, those elements may be combined in other ways to accomplish the same objectives. Acts, elements and features discussed only in connection with one embodiment are not intended to be excluded from a similar role in other embodiments or embodiments.

The apparatuses described herein may be embodied in other specific forms without departing from the characteristics thereof. Scope of the apparatuses described herein is thus indicated by the appended claims, rather than the foregoing description, and changes that come within the meaning and range of equivalence of the claims are embraced therein.

### LIST OF REFERENCE SIGNS

10: aerosol generation device
12: body of aerosol generation device
14: mouthpiece
100: controller
110: orientation sensor
120: heating arrangement
130: memory

1. An aerosol generation device for generating an aerosol or vapor, the aerosol generation device comprising:
an orientation sensor for sensing an orientation of the aerosol generation device, and
a controller for:
detecting a first set of one or more states of the aerosol generation device, the first set comprising at least one state detected based at least in part on the sensed orientation;
determining, based at least in part on the first set of states, a first operation of the aerosol generation device to be performed;
controlling the aerosol generation device in accordance with the first operation; and
causing a memory to store the first set of states and the first operation in association with each other, the association being for controlling a subsequent operation of the aerosol generation device when a subsequently detected second set of states of the aerosol generation device matches the first set of states.

2. The aerosol generation device according to claim 1, wherein the controller is configured to determine the first operation based on:
an association between the first set of states and the first operation stored in the memory;
a difference between a value of a parameter indicated by the first set of states and a predetermined value of the parameter; or
a state of the first set of states detected at least in part based on a user action indicating the first operation.

3. The aerosol generation device according to claim 1, wherein
the memory is configured for storing at least one predetermined set of states of the aerosol generation device, each predetermined set comprising one or more respective states of the aerosol generation device, and at least one predetermined operation of the aerosol generation device to be performed, each predetermined operation being to be associated with a set of states stored in memory,
wherein the controller is configured for:
determining a predetermined set of states matching the first set of states, and
controlling the aerosol generation device to operate in accordance with the predetermined operation associated with the predetermined set of states.

4. The aerosol generation device according to claim 1, wherein the controller is configured for:
determining a third set of states stored in the memory that matches the first set of states,
determining a difference between a state of the first set of states and a corresponding state of the third set of states,
determining a change in operation of the aerosol generation device required to decrease the difference in a subsequent operation of the aerosol generation device, and
cause the memory to store data defining the change of operation in association with data defining a subset of the third set of states.

5. The aerosol generation device according to claim 1, wherein the controller is configured for causing, in case the memory already stores the first set of states and/or the memory already stores the first operation, the memory to store an association between the first set of states and the first operation.

6. The aerosol generation device according to claim 1, wherein the controller is configured for detecting a movement of the aerosol generation device based on the sensed orientation,
wherein the first set of states comprises at least one state based on the detected movement.

7. The aerosol generation device according to claim 1, wherein
the controller is configured for detecting an action of a user on the aerosol generation device, wherein,
the first set of states comprises at least one state based on the detected action.

8. The aerosol generation device according to claim 1, further comprising
a heating arrangement for heating the aerosolisable substance; and
an electrical power supply,
wherein the controller is configured for controlling, as one of the at least one operation, a supply of electrical power from the electrical power supply to the heating arrangement.

9. The aerosol generation device according to claim 1, further comprising a thermal sensor for sensing a thermal characteristic of the aerosol generation device and/or the aerosolisable substance,
wherein the first set of states comprises at least one state based on a thermal value output from the thermal sensor.

10. The aerosol generation device according to claim 9, wherein the controller is configured for determining an allowable range for the thermal value based on the sensed orientation, and for reducing or interrupting, as part of the first operation, the heating of the aerosolisable substance and/or the generation of aerosol if the thermal value does not fall within the allowable range.

11. The aerosol generation device according to claim 1, further comprising a second sensor for sensing at least one of an atmospheric pressure and an altitude of the aerosol generation device,
wherein the detected set of states comprises at least one state of the aerosol generation device based on a sensed atmospheric pressure or altitude.

12. The aerosol generation device according to claim 1, further comprising a power source,
wherein the controller is configured for detecting a state of the power source, and
wherein the first set of states comprises at least one state determined based on a detected state of the power source.

13. The aerosol generation device according to claim 1, wherein the first operation includes interrupting the operation of the aerosol generation device.

14. An aerosol generation device according to claim 1, wherein the orientation sensor comprises at least one of an accelerometer and a gyroscope.

15. An aerosol generation device according to claim 1, wherein the orientation sensor is configured to sense the orientation of the aerosol generation device in a three dimensional space.

16. A controller for an aerosol generation device, the aerosol generation device being for generating an aerosol or vapor and comprising an orientation sensor for sensing an orientation of the aerosol generation device,
wherein the controller is configured for:
detecting a first set of one or more states of the aerosol generation device, the first set comprising at least one state detected based at least in part on the sensed orientation;
determining, based at least in part on the first set of states, a first operation of the aerosol generation device to be performed;
controlling the aerosol generation device in accordance with the first operation; and
causing a memory to store the first set of states and the first operation in association with each other, the association being for controlling a subsequent operation of the aerosol generation device when a subsequently detected second set of states of the aerosol generation device matches the first set of states.

17. A method for controlling an aerosol generation device, the aerosol generation device being for generating an aerosol or vapor and comprising an orientation sensor for sensing an orientation of the aerosol generation device, the method comprising:
detecting a first set of one or more states of the aerosol generation device, the first set comprising at least one state detected based at least in part on the sensed orientation;
determining, based at least in part on the first set of states, a first operation of the aerosol generation device to be performed;
controlling the aerosol generation device in accordance with the first operation; and
causing a memory to store the first set of states and the first operation in association with each other, the association being for controlling a subsequent operation of the aerosol generation device when a subsequently detected second set of states of the aerosol generation device matches the first set of states.

18. A computer program comprising instructions which, when executed by one or more processor, cause the one or more processors to perform the method according to claim 17.

19. An aerosol generation device for generating an aerosol or vapor, the aerosol generation device comprising:
an orientation sensor for sensing an orientation of the aerosol generation device;
a memory for storing a machine learning model having, as an input, a set of states of the aerosol generation device, and as an output, at least one operation of the aerosol generation device to be performed; and
a controller for:
detecting a set of states of the aerosol generation device, the detected set of states comprising at least one state detected based at least in part on the sensed orientation;
determining, based at least in part on the detected set of states, an operation of the aerosol generation device to be performed;
controlling an operation of the aerosol generation device in accordance with the determined operation; and
causing the machine learning model to learn an association between the detected set of states and the determined operation.
